(19) United States
(12) Patent Application Publication
(10) Pub. No.: US 2019/0142359 A1
(43) Pub. Date: May 16, 2019

(54) SURGICAL POSITIONING SYSTEM AND POSITIONING METHOD

(71) Applicant: TINAVI MEDICAL TECHNOLOGIES CO., LTD., Beijing (CN)

(72) Inventors: Songgen ZHANG, Beijing (CN); Wei TIAN, Beijing (CN); Yajun LIU, Beijing (CN); Jin XU, Beijing (CN); Weijun ZHANG, Beijing (CN); Binbin WANG, Beijing (CN)

(73) Assignee: TINAVI MEDICAL TECHNOLOGIES CO., LTD., Beijing (CN)

(21) Appl. No.: 16/307,475

(22) PCT Filed: Oct. 27, 2016
(86) PCT No.: PCT/CN2016/103503

§ 371 Date: Dec. 5, 2018

(30) Foreign Application Priority Data
Jun. 8, 2016 (CN) 2016104039847

(51) Int. Cl. A61B 6/00; A61B 34/20

(52) U.S. Cl. CPC A61B 6/584; A61B 34/30; A61B 34/20

(57) ABSTRACT

The present disclosure relates to a surgical positioning system and a positioning method. The surgical positioning system comprises a surgical robot, a host computer, a spatial measurement device, a robot tracer, a three-dimensional imaging device and a calibrator for three-dimensional image. The host computer is configured to control a motion of the surgical robot. The calibrator and the robot tracer are detachably connected to a terminal end of the surgical robot. The spatial measurement device is configured to measure spatial coordinates of the robot tracer and transmit position data to the host computer. The three-dimensional imaging device is configured to scan the calibrator and a surgical site of the patient and transmit an image of the markers and an image of the patient to the host computer. The host computer is configured to identify and match the markers in the image and the markers on the calibrator.

### CROSS REFERENCE

The present disclosure claims the benefit of an International Patent Application No. PCT/CN2016/103503 filed on Oct. 27, 2016, which claims the benefit of a Chinese Patent Application No. 201610403984.7 filed on Jun. 8, 2016. The above patent applications are incorporated entirely by reference in the disclosure.

### TECHNICAL FIELD

The present disclosure relates to a surgical positioning system and a positioning method, which belong to the technical field of surgical navigation.

### BACKGROUND

With the widespread application of minimally invasive surgery and the continuously increasing requirements for the precision of positioning of instruments or implants in surgery in recent years, auxiliary positioning or surgical navigation systems based on medical image guidance have made great progress. The implementation of such systems generally includes several steps: first, space calibrating and image registration. That is, a spatial transformation relationship between coordinate systems of a surgical target (patient), images of the target, and an auxiliary positioning device is calculated through a spatial coordinate calibrating method. The step generally is referred to as multi-coordinate system calibration or image registration. The next step is surgical planning and guidance. That is, a preoperative or intraoperative image having an accurate calibration is displayed, and a doctor plans a surgery path on the image or on a re-constructed three-dimensional model. Subsequently, the next step is surgical implementation, which mainly involves surgery path positioning, that is, guiding a doctor to place a surgical tool guiding device onto the surgery path by hands or to directly control an execution mechanism such as a robotic arm, to accurately place a guiding device onto the surgery path, so as to guarantee the precision of surgery path guidance, and the doctor implements operations, such as surgical instruments implantation, by means of the guiding device.

Among the foregoing steps, the step of spatial calibrating and image registration is an extremely significant step. The step generally means standardizing multiple coordinate systems (generally including an image coordinate system, a tool (auxiliary positioning apparatus) coordinate system, and a patient coordinate system) into one same coordinate system in an image guidance-based surgical positioning system. The process is also referred to as registration or calibration. The precision of the registration determines the precision of the auxiliary positioning or surgical navigation.

According to types of medical images used (fluoroscopy images, or three-dimensional images) and sources of the medical images (preoperative images, or intraoperative images obtained on site), the commonly used image registration methods at present are as follows.

Scenario 1: the requirement for image registration is “obtaining three-dimensional images before a surgery and doing images registration during the surgery”.

Methods for image registration that meet the requirement for image guided surgery are described below. In method (1), during a surgery, some anatomical feature points of a human body are detected with a spatial coordinate measurement device and then paired with corresponding feature points in an image to implement image registration. In method (2), during a surgery, coordinate information corresponding to a feature contour of a human body is continuously obtained by using a spatial coordinate measurement device, and then paired with information on corresponding positions and shapes in preoperative images in a point set registration process, to implement image registration. In method (3), preoperative three-dimensional images of a patient are obtained with several markers attached on the patient outside of his/her surgical site. During the surgery, coordinates of a marker are obtained by using a spatial coordinate measurement device, and meanwhile, a corresponding marker in the image is paired with the coordinates and marked. Repeat the above process for respective markers at different positions to implement image registration.

Scenario 2: the requirement for image registration is “obtaining three-dimensional images before a surgery and spatial calibrating them with fluoroscopy images obtained during the surgery”.

A method to meet the requirement for image registration includes: identifying and matching a contour or an edge shape of an anatomical structure in an intraoperative fluoroscopy image with that in a preoperative three-dimensional image by using a special algorithm, to implement registration from the preoperative three-dimensional image to the intraoperative fluoroscopy image.

Scenario 3: the requirement for image registration is “obtaining a 2D fluoroscopy image during a surgery and registering on site”.

A method for image registration that meets the requirement is described below. A patient tracer and a robot tracer are traced by a spatial coordinate measurement device, wherein the patient tracer is fixedly mounted on a patient body. A dual-parallel, planar-structured special calibrator is mounted at a terminal end of a robotic arm, and the robot tracer is mounted on the robotic arm. During a surgery, fluoroscopy images are obtained from at least two different angles, and intraoperative fluoroscopy image registration is implemented by identifying calibrator markers in the image.

Scenario 4: the requirement for image registration is “obtaining a set of three-dimensional images during the surgery and doing image registration on site”.

A method that meets the requirement is described below. A spatial coordinate measurement device detects coordinate information of an intraoperative three-dimensional imaging device (CT or MRI or C-arm with three-dimensional option). Coordinate information of a patient is obtained according to patient tracers installed on the patient's body or a place relatively stationary with respect to the patient's body. A spatial transform relationship (a rotation and translation matrix) between the intraoperative three-dimensional image coordinate system and the patient coordinate system is calculated by calibration or by means of parameters in an imaging device provided by the imaging device manufacturer, to implement intraoperative three-dimensional image registration.

The method in scenario 4 depends on a tracer mounted on an intraoperative imaging device, and meanwhile a series of imaging parameters of the imaging device need to be calibrated in advance; and therefore, the method is not easy to implement.

### SUMMARY

With respect to the above problem, an object of the disclosure is to provide a calibrator for three-dimensional image, a surgical positioning system and a positioning method. The positioning method is capable of implementing automatically intraoperative three-dimensional image registration independent of parameters of a three-dimensional imaging device, and is easy to implement.

To achieve the object, the present disclosure provides a calibrator for three-dimensional image, characterized in that: the calibrator for three-dimensional image comprises a calibrator plane and a calibrator handle, wherein the calibrator plane is flat or arc-shaped, and at least four markers to be identified by a three-dimensional imaging device are arranged on the calibrator plane; and one end of the calibrator handle is fixedly connected to the calibrator plane, and a connector for connecting to a surgical robotic arm is provided at the other end of the calibrator handle.

All markers are anisotropically arranged on the calibrator plane.

The calibrator plane is made of an X-ray transparent material; and the markers are made of an X-ray opaque material.

The present disclosure further provides a surgical positioning system, characterized in that: the surgical positioning system comprises a surgical robot, a host computer, a spatial measurement device, a robot tracer, a patient tracer, a three-dimensional imaging device, and a calibrator for three-dimensional image; the surgical robot is a robotic arm having at least three translational degrees of freedom and three rotational degrees of freedom; the host computer is electrically connected to the surgical robot so as to control a motion of the surgical robot; the calibrator for three-dimensional image and the robot tracer are configured to be detachably connected to a terminal end of the surgical robot; the patient tracer is configured to be fixed on a patient's body; the spatial measurement device is configured to measure spatial coordinates of the robot tracer and the patient tracer and transmit position data to the host computer; the three-dimensional imaging device is configured to scan the calibrator for three-dimensional image and a surgical site of the patient and transmit an image of the markers and an image of the patient to the host computer; and the host computer is configured to identify and match the markers in the image and the markers on the calibrator for three-dimensional image.

The surgical positioning system further comprises a guiding device, wherein the guiding device is configured to be detachably connected to the terminal end of the surgical robot.

The present disclosure further provides a positioning method, comprising the following steps: (1) placing a calibrator for three-dimensional image, installed on a surgical robot, close to a surface of a patient's body at a surgical site; scanning both the calibrator and the surgical site of the patient with a three-dimensional imaging device; obtaining, with the three-dimensional imaging device, three-dimensional images of markers on the calibrator and the patient, and transmitting the images to the host computer; and tracking, with a spatial measurement device, coordinates of a robot tracer and a patient tracer, and transmitting the coordinates to the host computer; (2) repeatedly comparing, with the host computer, geometric features of the markers in the image and preset geometric features of these markers, to identify and match the markers on the calibrator for three-dimensional image and the markers in the image; (3) calculating, with the host computer, a coordinate transformation relationship between the patient image and the robot tracer according to a given coordinate relationship between the markers on the calibrator for three-dimensional image and the robot tracer, and further calculating a coordinate transformation relationship between the patient image and the surgical robot; and (4) calculating a coordinate of a spatial point in a robot coordinate system that corresponds to any point in the patient image, according to the coordinate transformation relationship between the patient image and the surgical robot, and further calculating coordinates of a surgery path that is determined in the patient image, in the robot coordinate system.

In step (2), the process of identifying the markers on the calibrator for three-dimensional image and the markers in the image comprises the following steps: (a) dividing the markers on the calibrator for three-dimensional image into a group A and a group B, wherein each group comprises three or more markers; (b) reading information about the markers in the group A and the group B in step (a) and information about the calibrator for three-dimensional image 1, and reading the images obtained by scanning in step (1); (c) performing threshold segmentation on the images obtained in step (b) and extracting and generating valid polygon data; (d) fitting and determining the polygon data obtained in step (c) according to the information about the calibrator for three-dimensional image obtained in step (b), so as to screen out markers in the image; (e) calculating a distance between each two markers among the markers in the image obtained in step (d); (f) selecting three markers from calibrator markers in the group A to construct a triangle as a triangular template, and searching for a triangle in the image that is approximately identical to the triangular template; if there is no such triangle, selecting three markers from calibrator markers in the group B to construct a triangle as a triangular template, and searching for a triangle in the image that is approximately identical to the triangular template; and if there is still no such triangle, selecting calibrator markers from the group A and the group B to construct a triangle as a triangular template, and searching for a triangle in the image that is approximately identical to the triangular template; and (g) matching serial numbers of respective vertices of the paired congruent triangles according to a one-to-one correspondence, to form a matching vertex pair, and searching for an image marker outside of the triangular template in the image corresponding to a calibrator marker with reference to the congruent triangular template, until all image markers match the calibrator markers.

The present disclosure adopts the foregoing technical solutions, and therefore has the following advantages. The present disclosure implements high-precision fusion or registration of a patient coordinate system, an image coordinate system, and a robot coordinate system, by using a calibrator for three-dimensional image and by means of a spatial measurement device, a patient tracer, and a robot tracer. The present disclosure performs vertex pair identification and marking without manual intervention, thereby having a high automation degree, independent of a special support of a three-dimensional imaging device, and having a wide applicability.

### BRIEF DESCRIPTION OF THE DRAWINGS

The disclosure will hereafter be described with reference to the accompanying drawings. It should be understood, however, that the accompanying drawings provide a better understanding of the present disclosure and are not meant to limit the scope of the disclosure.

FIG. 1 is a schematic structural diagram of a calibrator for three-dimensional image according to the disclosure.

FIG. 2 is a schematic structural diagram of a surgical positioning system according to the disclosure.

FIG. 3 is a schematic structural diagram of a guiding device according to the disclosure.

### DETAILED DESCRIPTION

The disclosure is described in detail below in the embodiments in combination with the accompanying drawings.

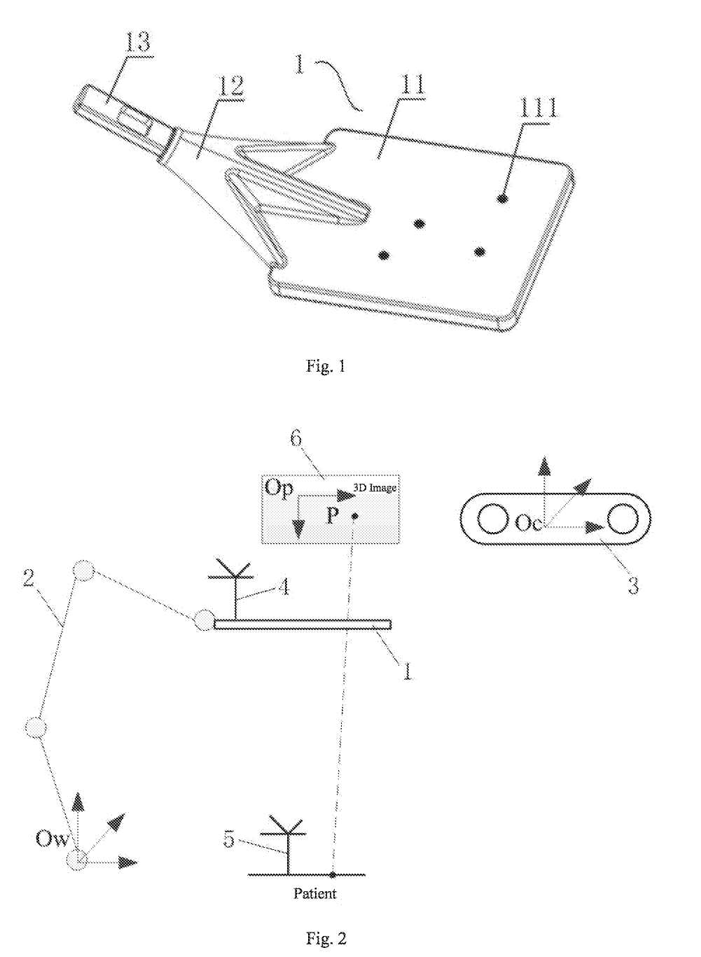

As shown in FIG. 1, the disclosure provides a calibrator for three-dimensional image 1. The calibrator for three-dimensional image 1 includes a calibrator plane 11 and a calibrator handle 12. The calibrator plane 11 is flat or arc-shaped. At least four markers 111 are arranged on the calibrator plane 1. The markers 111 are configured to be identified and scanned by a three-dimensional imaging device to form an image. One end of the calibrator handle 12 is fixedly connected to the calibrator plane 11, and a connector 13 for connecting to the surgical robotic arm is provided at the other end of the calibrator handle 12.

Further, all markers 111 are anisotropically arranged on the calibrator plane 1 (for example, any two distances between the markers 111 are not equal).

Further, the calibrator plane 1 is made of an X-ray transparent material; and the markers 111 are made of an X-ray opaque material.

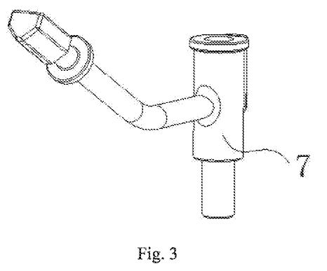

As shown in FIG. 2, based on the above calibrator for three-dimensional image 1, the disclosure further provides a surgical positioning system. The surgical positioning system includes a calibrator for three-dimensional image 1, a surgical robot 2, a host computer (not shown), a spatial measurement device 3, a robot tracer 4, a patient tracer 5, a three-dimensional imaging device 6, and a guiding device 7. The surgical robot 2 is a robotic arm having at least three translational degrees of freedom and three rotational degrees of freedom. The host computer is electrically connected to the surgical robot 2 so as to control a motion of the surgical robot 2. The calibrator for three-dimensional image 1 and the robot tracer 4 are connected to a terminal end of the surgical robot through a quick-mount and quick-release device. The patient tracer 5 is fixed on a patient's body. The spatial measurement device 3 can measure spatial coordinates of the robot tracer 4 and the patient tracer 5, and updates the coordinates at a certain frequency, to implement real-time tracing. The spatial measurement device 3 can adopt a high-precision optic tracing camera based on stereo vision or may be based on other principles, and transmit position data to the host computer. The three-dimensional imaging device 6 is configured to scan the calibrator for three-dimensional image 1 so as to form an image of the markers 111. The host computer identifies and matches the markers in the image and the markers 111 on the calibrator for three-dimensional image 1. As shown in FIG. 3, the guiding device 7 is an apparatus for fixing a needle insertion path. The guiding device 7 is connected to the surgical robot 2 through a quick-mount and quick-release device, the same as that for the calibrator 1. The guiding device 7 and the calibrator for three-dimensional image 1 are alternatively mounted for use as needed in a surgery.

The present disclosure preferably adopts a cone-beam CT machine (CBCT machine) as the three-dimensional imaging device.

Based on the above positioning system, the disclosure provides a positioning method, which is applicable to spatial positioning of a surgery path. The method includes the following steps. Step (1) comprises: placing a calibrator for three-dimensional image 1, installed on a surgical robot 2, close to a surface of a patient's body at a surgical site (close to but not in contact with the surface); scanning both the calibrator for three-dimensional image 1 and the surgical site of the patient with a three-dimensional imaging device 6 (the three-dimensional image scanning is performed only once without fluoroscopy from more than one different angles for several times); obtaining, with the three-dimensional imaging device 6, three-dimensional images of markers 111 on the calibrator 1 and of the patient, and transmitting the images to a host computer; and tracking, with a spatial measurement device 3, coordinates of a robot tracer 4 and a patient tracer 5, and transmitting the coordinates to the host computer.

Step (2) comprises: repeatedly comparing, with the host computer, geometric features of the markers in the image and preset geometric features of these markers, to identify and match the markers 111 on the calibrator for three-dimensional image 1 and the markers in the image.

Step (3) comprises: calculating, with the host computer, a coordinate transformation relationship between the patient image and the robot tracer 4 according to a given coordinate relationship between the markers 111 on the calibrator for three-dimensional image 1 and the robot tracer 4 (it should be noted that the host computer may further calculate a coordinate transformation relationship between the patient image and the patient tracer 5 according to coordinates of the robot tracer 4 and the patient tracer 5 obtained by the spatial measurement device 3), and further calculating a coordinate transformation relationship between the patient image and the surgical robot 2. The step may also comprise: directly calculating, with the host computer, a coordinate transformation relationship between the patient image and the surgical robot 2 according to a given coordinate relationship between the markers 111 on the calibrator for three-dimensional image 1 and the surgical robot 2.

Step (4) comprises: calculating a coordinate of a spatial point in a robot coordinate system that corresponds to any point in the patient image, according to the coordinate transformation relationship between the patient image and the surgical robot 2 obtained in step (3). If the surgery path is represented by a straight line in the patient image, coordinates of the surgery path in the robot coordinate system can be calculated.

By means of dedicated software, a doctor may draw a surgery path on a registered image as needed in treatment. After spatial coordinates of the surgery path is calculated according to the spatial positioning method for the surgery path, the doctor may control the surgical robot 2 to move accurately so as to enable a guiding structure of the guiding device 7 that is connected to the terminal end of the surgery robot 2 to orient at the surgery path. In the foregoing process, the spatial measurement device 3 having a real-time tracing function monitors the patient tracer 5 (that is, a movement of the patient) in real time, and calculates an orientation and magnitude of the movement. The surgical robot 2 may modify its own motion according to data such as the orientation and magnitude of the movement, so as to guarantee that the guiding device precisely conforms to the planned surgery path.

In step (2), the specific process of identifying the markers 111 on the calibrator for three-dimensional image 1 and the markers in the image comprises the following substeps.

Substep (a) comprises: dividing the markers 111 on the calibrator for three-dimensional image 1 into a group A and a group B, wherein each group includes three or more markers 111.

Substep (b) comprises: reading information about the markers in the group A and the group B in substep (a) and information about the calibrator for three-dimensional image 1, and reading the images obtained by scanning in step (1).

Substep (c) comprises: performing threshold segmentation on the images obtained in substep (b) and extracting and generating valid polygon data.

Substep (d) comprises: fitting and determining the polygon data obtained in substep (c) according to the information about the calibrator for three-dimensional image 1 obtained in substep (b), so as to screen out markers in the image.

Substep (e) comprises: calculating a distance between each two markers among the markers in the image obtained in substep (d).

Substep (f) comprises: selecting three markers from calibrator markers in the group A to construct a triangle as a triangular template, and searching for a triangle in the image that is approximately identical to the triangular template; if there is no such triangle, selecting three markers from calibrator markers in the group B to construct a triangle as a triangular template, and searching for a triangle in the image that is approximately identical to the triangular template; and if there is still no such triangle, selecting calibrator markers from the group A and the group B to construct a triangle as a triangular template, and searching for a triangle in the image that is approximately identical to the triangular template.

Substep (g) comprises: matching serial numbers of respective vertices of the paired congruent triangles according to a one-to-one correspondence, to form a matching vertex pair, and searching for an image marker outside of the triangular template in the image corresponding to a calibrator marker with reference to the congruent triangular template, until all image markers match the calibrator markers.

The foregoing embodiments are used to describe the present disclosure only, and the structures, the disposing positions, and the connections of all the components can be different. Modifications or equivalent alternations made to a specific component according the principles of the present disclosure on the basis of the technical solutions of the present disclosure should fall within the protection scope of the present disclosure.

1. (canceled)

2. (canceled)

3. (canceled)

4. A surgical positioning system, comprising a surgical robot, a host computer, a spatial measurement device, a robot tracer, a three-dimensional imaging device, and a calibrator for three-dimensional image;
the host computer is electrically connected to the surgical robot so as to control a motion of the surgical robot;
the calibrator for three-dimensional image comprises a calibrator plane and a calibrator handle, wherein the calibrator plane is flat or arc-shaped, and at least four markers to be identified by a three-dimensional imaging device are arranged on the calibrator plane; and one end of the calibrator handle is fixedly connected to the calibrator plane, and a connector for connecting to a surgical robotic arm is provided at the other end of the calibrator handle;
the calibrator for three-dimensional image and the robot tracer are configured to be detachably connected to a terminal end of the surgical robot;
the spatial measurement device is configured to measure spatial coordinates of the robot tracer and transmit position data to the host computer;
the three-dimensional imaging device is configured to scan the calibrator for three-dimensional image and a surgical site of the patient and transmit an image of the markers and an image of the patient to the host computer; and
the host computer is configured to identify and match the markers in the image and the markers on the calibrator for three-dimensional image.

5. The surgical positioning system according to claim 4, further comprising a guiding device, wherein the guiding device is configured to be detachably connected to the terminal end of the surgical robot.

6. A surgical positioning method, comprising the following steps:
(1) placing a calibrator for three-dimensional image, installed on a surgical robot, close to a surface of a patient's body at a surgical site, wherein the calibrator for three-dimensional image comprises a calibrator plane and a calibrator handle, wherein the calibrator plane is flat or arc-shaped, and at least four markers to be identified by a three-dimensional imaging device are arranged on the calibrator plane, and one end of the calibrator handle is fixedly connected to the calibrator plane, and a connector for connecting to a surgical robotic arm is provided at the other end of the calibrator handle; scanning both the calibrator and the surgical site of the patient with a three-dimensional imaging device; obtaining, with the three-dimensional imaging device, three-dimensional images of markers on the calibrator and the patient, and transmitting the images to a host computer; and tracking, with a spatial measurement device, coordinates of a robot tracer, and transmitting the coordinates to the host computer, wherein the robot tracer is configured to be detachably connected to a terminal end of the surgical robot;
(2) repeatedly comparing, with the host computer, geometric features of the markers in the image and preset geometric features of these markers, to identify and match the markers on the calibrator for three-dimensional image and the markers in the image;
(3) calculating, with the host computer, a coordinate transformation relationship between the patient image and the surgical robot; and
(4) calculating, with the host computer, a coordinate of a spatial point in a robot coordinate system that corresponds to any point in the patient image, according to the coordinate transformation relationship between the patient image and the surgical robot.

7. The surgical positioning method according to claim 6, wherein in step (2), the process of identifying the markers on the calibrator for three-dimensional image and the markers in the image comprises the following steps:
(a) dividing the markers on the calibrator for three-dimensional image into a group A and a group B, wherein each group comprises three or more markers;
(b) reading information about the markers in the group A and the group B in step (a) and information about the calibrator for three-dimensional image 1, and reading the images obtained by scanning in step (1);
(c) performing threshold segmentation on the images obtained in step (b) and extracting and generating valid polygon data;
(d) fitting and determining the polygon data obtained in step (c) according to the information about the calibrator for three-dimensional image obtained in step (b), so as to screen out markers in the image;
(e) calculating a distance between each two markers among the markers in the image obtained in step (d);
(f) selecting three markers from calibrator markers in the group A to construct a triangle as a triangular template, and searching for a triangle in the image that is approximately identical to the triangular template; if there is no such triangle, selecting three markers from calibrator markers in the group B to construct a triangle as a triangular template, and searching for a triangle in the image that is approximately identical to the triangular template; and if there is still no such triangle, selecting calibrator markers from the group A and the group B to construct a triangle as a triangular template, and searching for a triangle in the image that is approximately identical to the triangular template; and
(g) matching serial numbers of respective vertices of the paired congruent triangles according to a one-to-one correspondence, to form a matching vertex pair, and searching for an image marker outside of the triangular template in the image corresponding to a calibrator marker with reference to the congruent triangular template, until all image markers match the calibrator markers.

8. The surgical positioning system according to claim 4, wherein all markers are anisotropically arranged on the calibrator plane.

9. The surgical positioning system according to claim 4, wherein the calibrator plane is made of an X-ray transparent material; and the markers are made of an X-ray opaque material.

10. The surgical positioning system according to claim 4, wherein the surgical robot is a robotic arm having at least three translational degrees of freedom and three rotational degrees of freedom.

11. The surgical positioning system according to claim 4, wherein the three-dimensional imaging device is a cone-beam CT machine.

12. The surgical positioning method according to claim 6, wherein in step (3), the host computer calculates a coordinate transformation relationship between the patient image and the robot tracer according to a given coordinate relationship between the markers on the calibrator for three-dimensional image and the robot tracer, and further calculates the coordinate transformation relationship between the patient image and the surgical robot.

13. The surgical positioning method according to claim 6, wherein in step (3), the host computer calculates the coordinate transformation relationship between the patient image and the surgical robot according to a given coordinate relationship between the markers on the calibrator for three-dimensional image and the surgical robot.

14. The surgical positioning method according to claim 6, further comprising: tracking, with the spatial measurement device, coordinates of a patient tracer, and transmitting the coordinates to the host computer, wherein the patient tracer is fixed on the patient's body.

15. The surgical positioning method according to claim 14, wherein in step (3), the host computer calculates a coordinate transformation relationship between the patient image and the patient tracer according to coordinates of the robot tracer and the patient tracer obtained by the spatial measurement device.

16. The surgical positioning method according to claim 6, wherein step (4) further comprises: calculating coordinates of a surgery path that is determined in the patient image, in the robot coordinate system.

17. The surgical positioning method according to claim 14, further comprising: monitoring in real-time and transmitting, with the spatial measurement device, a movement of the patient tracer to the host computer; and calculating, with the host computer, an orientation and magnitude of the movement and controlling the surgical robot to modify its motion according to the orientation and magnitude of the movement.

18. A surgical positioning system, comprising a surgical robot, a host computer, a spatial measurement device, a robot tracer, a patient tracer, a three-dimensional imaging device, and a calibrator for three-dimensional image;
the host computer is electrically connected to the surgical robot so as to control a motion of the surgical robot;
the calibrator for three-dimensional image comprises a calibrator plane and a calibrator handle, wherein the calibrator plane is flat or arc-shaped, and at least four markers to be identified by a three-dimensional imaging device are arranged on the calibrator plane; and one end of the calibrator handle is fixedly connected to the calibrator plane, and a connector for connecting to a surgical robotic arm is provided at the other end of the calibrator handle;
the calibrator for three-dimensional image and the robot tracer are configured to be detachably connected to a terminal end of the surgical robot;
the patient tracer is configured to be fixed on a patient's body;
the spatial measurement device is configured to measure spatial coordinates of the robot tracer and the patient tracer and transmit position data to the host computer;
the three-dimensional imaging device is configured to scan the calibrator for three-dimensional image and a surgical site of the patient and transmit an image of the markers and an image of the patient to the host computer; and
the host computer is configured to identify and match the markers in the image and the markers on the calibrator for three-dimensional image.

19. The surgical positioning system according to claim 18, wherein all markers are anisotropically arranged on the calibrator plane.

20. The surgical positioning system according to claim 18, wherein the calibrator plane is made of an X-ray transparent material; and the markers are made of an X-ray opaque material.

21. The surgical positioning system according to claim 18, further comprising a guiding device, wherein the guiding device is configured to be detachably connected to the terminal end of the surgical robot.

22. The surgical positioning system according to claim 18, wherein the surgical robot is a robotic arm having at least three translational degrees of freedom and three rotational degrees of freedom.

23. The surgical positioning system according to claim 18, wherein the spatial measurement device is configured to monitor in real-time and transmit a movement of the patient tracer to the host computer, and the host computer is configured to calculate an orientation and magnitude of the movement and control the surgical robot to modify its motion according to the orientation and magnitude of the movement.
